(19) United States
(12) Patent Application Publication
(10) Pub. No.: US 2009/0227023 A1
(43) Pub. Date: Sep. 10, 2009

(54) DE-DIFFERENTIATION OF ASTROCYTES INTO NEURAL STEM CELL USING Shh

(76) Inventors: Seungkwon You, Gyeonggi-do (KR); Jai Hee Moon, Seoul (KR); Byung Sun Yoon, Seoul (KR); Ki Dong Kim, Gyeonggi-do (KR); Gyuman Park, Gyeonggi-do (KR); Seung Jun Yoo, Gyeonggi-do (KR); Eun Kyung Jun, Gyeonggi-do (KR); Bona Kim, Seoul (KR); Sung Sik Kwak, Incheon (KR); Isaac Maeng, Seoul (KR)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/280,647

(22) PCT Filed: Apr. 12, 2006
(86) PCT No.: PCT/KR2006/001349

§ 371 Date: Sep. 3, 2008

(30) Foreign Application Priority Data
Feb. 27, 2006 (KR) 10-2006-0019014

(51) Int. Cl. C12N 5/02

(52) U.S. Cl. 435/377

(57) ABSTRACT

Disclosed are a composition and a method for inducing the de-differentiation of astrocytes into neural stem cells using Shh. The de-differentiated neural stem cells have the ability to differentiate into astrocytes, neurons, and oligodendrocytes.

### TECHNICAL FIELD

Neural stem cells (NSCs) are a subtype of progenitor cells in the nervous system, which have the ability to differentiate into astrocytes, oligodendrocytes, and neurons. Originating from the Central Nervous System (CNS) and the Peripheral Nervous System (PNS), neural stem cells form multicellular neurospheres, which differentiate into glial lineage and neural lineage cells under respective sets of conditions (Sally Temple et al. 2001). These neural stem cells are used in the treatment of incurable diseases, and are being studied for potential use in cell treatment methods. Extensive research has been conducted on neural stem cells because they are adult stem cells that entail few ethical problems. Active research on de-differentiation, which has been conducted recently, is increasing the importance of adult stem cells. Adult stem cells are easier to obtain than embryonic stem cells, but there are still many difficulties in the practical application thereof. In addition, immune rejection response can be a problem when using adult stem cells from other people. Therefore, the induction of de-differentiation using a patient's own cells could solve current problems. For this purpose, it is necessary to induce the de-differentiation of cells that are already differentiated. Currently, extensive research is underway to induce de-differentiation using methods such as cell fusion and nuclear transfer. In another group using different methods, Alexis J. reported that cells isolated from the skin have the characteristics of neural stem cells when cultured under neural stem cell cultivation conditions (Lancet 2004). In addition, Toru K. succeeded in de-differentiating oligodendrocyte precursors into neural stem cells (Genes & Development 2004). This group has been publishing papers on this issue since 2000, and reported in a 2004 paper that gene expression at each stage is relevant to chromatin remodeling and histone modification.

### BACKGROUND ART

In the present invention, a solution to the problems of formerly introduced methods is sought, and a novel proper usage method is established. In this invention, Sonic Hedgehog (Shh), a member of the hedgehog gene family that plays an important role in development, is used because it acts as a morphogen having a high influence on neural stem cells. Shh is an upregulation factor of the signaling pathway, which initiates from Gli1, with Bmi-1 and N-myc acting as subsequent mediators. Upon development, Shh is expressed in distal tips of the follicular epithelium. Also, Shh is known to promote the proliferation of neural progenitor cells (Pleasantin Mill et al., 2005).

Leading to the present invention, intensive and thorough research conducted by the present inventors with this background, resulted in the finding that treatment with Shh induces already differentiated astrocytes to de-differentiate into neural stem cell-like cells capable of self-renewal and differentiation into astrocytes, neurons, and oligodendrocytes.

### DISCLOSURE OF THE INVENTION

An object of the invention is to provide a composition for inducing the de-differentiation of astrocytes into neural stem cells, comprising an Shh protein or a nucleic acid material containing a nucleotide sequence coding for an Shh protein.

Another object of the invention is to provide a method of inducing the de-differentiation of astrocytes into neural stem cells, comprising the treatment of astrocytes with an Shh protein or a nucleic acid material containing a nucleotide sequence coding for an Shh protein.

A further object of the invention is to provide neural stem cells which are produced using the method.

Still a further object of the invention is to provide a method of differentiating the de-differentiated neural stem cells into astrocytes, oligodendrocytes and neurons.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows the de-differentiation of astrocytes into neural stem cells through treatment with an Shh protein, in which neurospheres are formed after the addition of Shh to mouse astrocytes (A, B) and to neural stem cells (C);

FIG. 2 shows the expression of neural stem cell markers, as analyzed through immunocytochemistry; and

FIG. 3 shows the in vitro differentiation of the neural stem cells (A-C) and neural stem cell-like cells (D-G) into astrocytes, oligodendrocytes and neurons, respectly.

### BEST MODE FOR CARRYING OUT THE INVENTION

In accordance with an aspect thereof, the present invention relates to a composition capable of inducing the de-differentiation of astrocytes into neural stem cells, which comprises an Shh protein or a nucleic acid material containing a nucleotide sequence coding for an Shh protein.

As used herein, the term “neural stem cell-like cells” is intended to indicate multipotent stem cells de-differentiated from somatic cells and capable of differentiating into astrocytes, oligodendrocytes, and neurons. In the present invention, neural stem cell-like cells are also described as neural stem cells.

It is newly disclosed in the present invention that when Shh is overexpressed in astrocytes, although already differentiated, they can de-differentiate into multipotent neural stem cell-like cells.

In the present invention, Shh is provided in the form of a protein.

As long as it originates from mammals, such as humans, horses, sheep, pigs, goats, camels, antelopes, and dogs, any Shh may be used in the composition of the present invention. In addition, the Shh protein of the present invention, used for de-differentiation into neural cells, may be a wild-type or a variant thereof.

The term “Shh protein variant” is intended to refer to Shh proteins, occurring naturally or artificially, which are different in amino acid sequence from the wild-type due to the deletion, insertion, non-conservative substitution or conservative substitution of amino acids, or due to combinations thereof. The variant is a functional equivalent which has the same biological activity as the native protein although its physical and/or chemical properties are modified. Preferably, the variant is increased in structural stability to physical and chemical environments or in physiological activity.

The composition of the present invention is preferably an Shh protein. For example, a culture medium for inducing the de-differentiation of astrocytes into neural stem cells is treated with an Shh protein.

The Shh protein is used in an amount sufficient to induce de-differentiation. The effective amount of the Shh protein depends on factors well known in the art, including culture media and culture methods. In a practical example of the present invention, the Shh protein is used in an amount of 500 ng/ml.

In a preferable embodiment of the present invention, the Shh is in the form of a nucleic acid material comprising a nucleotide sequence coding for the Shh protein.

The nucleotide sequence that encodes an Shh protein is a wild-type or a variant thereof, which, naturally occurring or artificially synthesized, is different in amino acid sequence from the wild-type due to the deletion, insertion, non-conservative substitution or conservative substitution of bases, or due to combinations thereof.

The nucleotide sequence that encodes an Shh protein can be either a single or a double strand consisting of a DNA molecule (genome, cDNA) or an RNA molecule.

In a preferred embodiment of the present invention, the nucleic acid material containing a nucleotide sequence encoding an Shh protein is a vector that allows an Shh protein to be expressed.

The term “vector”, as used herein, is intended to refer to a DNA construct containing a DNA sequence which is operably linked to a control sequence capable of effecting the expression of the DNA in a suitable host cell.

The term “operably linked”, as used herein, is intended to refer to a functional linkage between a nucleic acid expression control sequence and a second nucleic acid sequence coding for a target protein in such a manner as to enable general functionality. The operable linkage to a recombinant vector may be prepared using a genetic recombinant technique that is well known in the art, and site-specific DNA cleavage and ligation may be carried out using enzymes that are generally known in the art.

A vector suitable for use in the present invention includes a signal sequence or a leader sequence for targeting membranes or secretion as well as expression regulatory elements, such as a promoter, an operator, an initiation codon, a stop codon, a polyadenylation signal and an enhancer, and can be constructed in various forms depending on the purpose thereof. The promoter of the vector may be constitutive or inducible. In addition, expression vectors include a selectable marker that allows the selection of host cells containing the vector, and replicable expression vectors include a replication origin. The vector may be self-replicable, or may be integrated into the DNA of a host cell.

The vector useful in the present invention may be a plasmid vector, a cosmid vector, or a viral vector, with preference for a viral vector. Examples of the viral vector includes vectors originated from retroviruses such as HIV (Human Immunodeficiency Virus), MLV (Murine Leukemia Virus), ASLV (Avian Sarcoma/Leukosis Virus), SNV (Spleen Necrosis Virus), RSV (Rous Sarcoma Virus), MMTV (Mouse Mammary Tumor Virus), etc., Adenovirus, Adeno-associated viruses, and Herpes Simplex virus, but are not limited thereto.

The nucleic acid material containing the nucleotide sequence coding for the Shh protein can be introduced into cells in the form of a vector as naked DNA (Wolff et al. Science, 247:1465-8, 1990: Wolff et al. J. Cell Sci. 103:1249-59, 1992), or with the aid of a liposome or a cationic polymer. For use in gene transfection, a liposome is a phospholipid membrane made of cationic phospholipids such as DOTMA and DOTAP. A cationic liposome, when mixed with a negatively charged nucleotide at a certain ratio, is formed into a nucleic acid-liposome complex.

In another preferred embodiment of the present invention, the nucleic acid material containing a nucleotide sequence that encodes the Shh protein may be a virus which expresses the Shh protein therein.

The term “virus”, as used herein, is intended to refer to an Shh-expressing virus which is prepared by transforming or transfecting a packaging cell with a viral vector carrying a nucleotide sequence coding for the Shh protein.

Examples of viruses useful in the preparation of the Shh-expressing viruses according to the present invention include retroviruses, adenoviruses, adeno-associated viruses, and the Herpes Simplex virus, but are not limited thereto.

In accordance with another aspect thereof, the present invention pertains to a method of de-differentiating astrocytes into neural stem cells, comprising treatment with an Shh protein or a nucleic acid material containing a nucleotide sequence encoding the Shh protein.

In greater detail, the method comprises the steps of (i) culturing astrocytes in a medium; (ii) treating the culture with an Shh protein or a nucleic acid material containing a nucleotide sequence coding for the Shh protein; and (iii) inducing the de-differentiation of astrocytes into neural stem cells.

Any conventional medium for culturing neural stem cells may be used as a medium for culturing astrocytes in step (i). Generally, a culture medium contains a carbon source, a nitrogen source, and trace element ingredients. In addition, the culture medium may include antibiotics, such as penicillin, streptomycin, and gentamicin. Preferred is a culture medium containing bFGF.

The Shh protein or the nucleic acid material containing the nucleotide sequence coding for the Shh protein with which the cells are treated in step (ii) is as mentioned above.

In accordance with a further aspect thereof, the present invention pertains to neural stem cells prepared in accordance with the aforementioned method.

It has been confirmed that the neural stem cells, prepared through the de-differentiation according to the present invention, express the neural stem cell-specific markers Nestin, CD133, and Sox2 at the same level, and have the same ability to differentiate as general neural stem cells. The neural stem cells prepared through de-differentiation according to the present invention also exhibit self-renewal.

In accordance with still a further aspect thereof, the present invention pertains to a method of differentiating the neural stem cells, de-differentiated according to the aforementioned method, into astrocytes, oligodendrocytes, and (or?) neurons.

When the neural stem cells, de-differentiated using the composition and method of the present invention, are placed under respective differentiation conditions suitable therefor, differentiation into astrocytes, neurons, and oligodendrocytes can be monitored by detecting the expression of markers specific for respective cells.

A better understanding of the present invention may be obtained through the following examples, which are set forth to illustrate, but are not to be construed as limiting the present invention.

### Example 1

### Experimental Method

1. Cultivation of Mouse Astrocytes and Mouse Neural Stem Cells

Mouse astrocytes, isolated from fetal brains at E13.5, were cultured in Dulbecco's modified Eagle's medium (DMEM (high glucose, HyClone)) supplemented with 10% FBS (HyClone), 1% penicillin/streptomycin, and 1% L-glutamine 1% (Cambrex). Neural stem cells were cultured as a control in Dulbecco's modified Eagle's medium/F12 (DMEM/F12, Gibco), supplemented with B27 serum-replacement (Gibco), human recombinant basic FGF and human recombinant EGF (R&D), containing (?) insulin (Sigma), apo-transferrin (Sigma), selenium (Sigma), progesterone (Sigma) and penicillin/streptomycin (Cambrex).

2. Induction of De-Differentiation

De-differentiation was induced under the same culture conditions used for neural stem cells. Cell culture was conducted using two different methods. Cells were plated at a density of 1×10⁵ cells/well in 6-well plates and incubated for 12 hrs, followed by treatment with Shh (sonic hedgehog, R&D) at a concentration of 500 ng/ml. The cells were cultured for 2, 4 and 6 days in the presence of Shh under conditions for the culture of astrocytes before the culture conditions were changed to be suitable for neural stem cells. Alternatively, the treatment with Shh was continued even after the culture conditions were changed.

3. Determination of Respective Markers Through Immunocytochemistry

After fixation with 4% paraformaldehyde (EMS) at 4° C. for one hour, neurospheres were incubated overnight in 20% sucrose with shaking. Then, the neurospheres were subjected to cryopreservation using an OCT compound in an 8-well chamber slide (Nunc) and sectioned 8˜10 μm thick before staining. After blocking with PBS containing 10% normal goat serum (Jackson ImmunoResearch)+0.1% BSA (Sigma)+0.3% Triton X-100 (Sigma), the sections were incubated with anti-nestin (Chemicon), anti-CD133 (MACS) and anti-Sox2 (Sigma) at 4° C. overnight and then with anti-mouse-cy3 (Jackson ImmunoResearch), anti-rabbit-FITC (Molecular probe), and anti-goat-cy3 (Zymed) at RT, finally followed by nuclear staining with DAPI (Sigma). A Zeiss confocal lens (Carl Zeiss) was used for examination after staining. Differentiated cells were stained in the same manner as described above. In this regard, anti-GFAP (Dako), anti-S100β(Zymed), anti-β-tubulin III (Covance), anti-Map2a (Sigma), anti-TH (Sigma), anti-04 (R&D), and anti-CNPase (Chemicon) were used as antibodies.

4. In Vitro Differentiation

After being coated with PLO (poly-L-ornithine) (Sigma) and laminin or fibronectin (Sigma), cells were cultured under the differentiation conditions given below.

For differentiation into astrocytes, cell culture was conducted for 5˜7 days in DMEM (HyClone, high glucose) supplemented with 10% FBS (HyClone) in the presence of human recombinant bFGF and EGF (R&D) or of CNTF (recombinant rat ciliary neurotrophic factor) (Upstate).

Differentiation into neurons was induced by culturing the cells in a medium containing N2 and B27 serum-replacement, supplemented with human recombinant FGF, for 4 days and then in an FGF-free medium for 8 days. Alternatively, cells were cultured for 7˜14 days in the presence of 1˜10 μM of RA (retinoic acid, Sigma) as a differentiation inducer. As an additional alternative, cells were cultured for 7˜14 days in the co-presence of 1 ˜10 mM of VPA (valproic acid, Sigma) and 1˜10 μM of RA (retinoic acid, Sigma) to induce differentiation into neurons.

Differentiation into oligodendrocytes was induced through incubation for 20 days in an N2 medium supplemented with B27 serum replacement in the presence of PDGF-AA (platelet derived growth factor-AA, R&D), T3 (3,3,5-triiodo-L-thyronine, Sigma), human recombinant basic FGF and EGF (R&D).

While being cultured under the aforementioned differentiation conditions, the cells were monitored for morphology, and immunocytochemistry using antibodies specific to respective differentiation markers was conducted to examine whether the differentiation proceeded accurately.

### Example 2

### Results

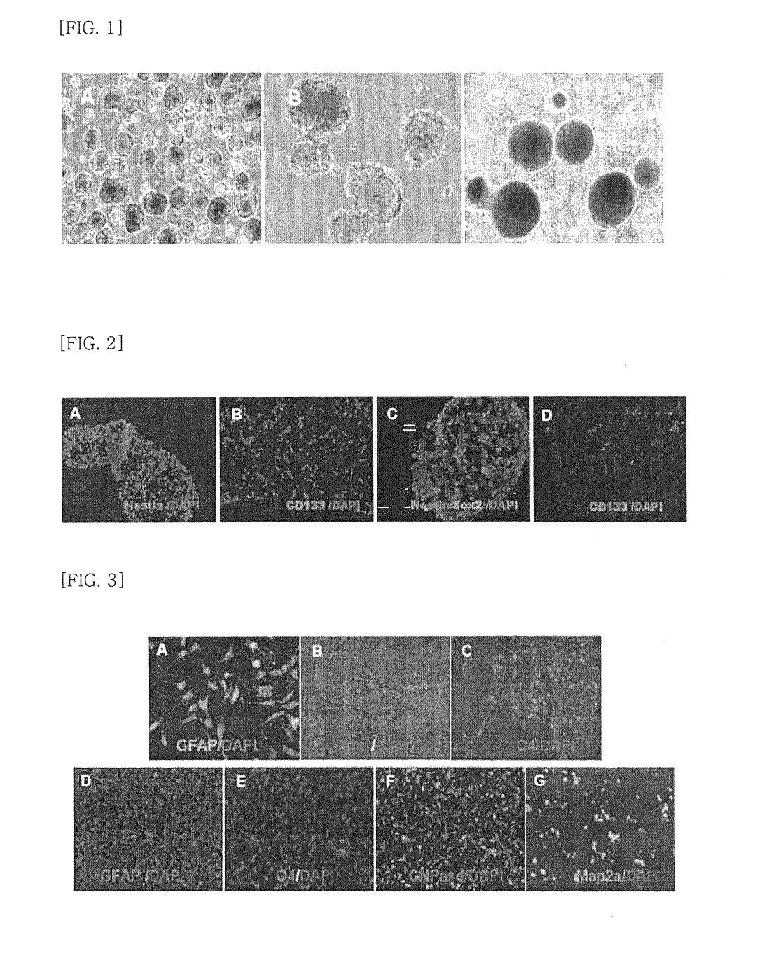

Mouse astrocytes, which had been differentiated, were treated with Shh, which acts to promote the proliferation of neural progenitor cells. Following the daily addition of Shh in an amount of 500 ng/ml to a medium for culturing mouse astrocytes, the culture medium was replaced with a medium for culturing neural stem cells. As the incubation proceeded, the cells were observed to have a morphology similar to that of neural stem cells. After the formation of neurospheres, no more Shh was added. Once formed, neurospheres persisted (FIG. 1).

The de-differentiation of mouse astrocytes was induced in the same manner. Following treatment with Shh for 2, 4 and 6 days, the culture conditions were changed to be suitable for neural stem cells, and the cells were monitored for de-differentiation. Starting from day 6 after treatment with Shh, neurospheres started to form better when the culture conditions were changed to be suitable for neural stem cells. The cells thus de-differentiated persisted.

In order to find out whether these cells had the same characteristics as neural stem cells, immunocytochemistry was performed using respective markers. In this regard, the markers nestin, CD133 and Sox2, which are most abundantly expressed in neural stem cells, were also observed in the neural stem cell-like cells after staining. Also, the cryo-section of the neurospheres thus formed ensured the expression of nestin, CD133 and Sox2 (FIG. 2).

In order to examine whether the neural stem cell-like cells could differentiate like neural stem cells, differentiation into astrocytes, oligodendrocytes and neurons was induced in the same manner as described above under several sets of conditions. The neural stem cell-like cells were found to differentiate into several types of cells, like neural stem cells, as analyzed using antibodies against the respective differentiation markers specific therefor. Identification was conducted with the expression of GFAP and S100 for differentiation into astrocytes, with the expression of β-tubulin III (Tuj1) and Map2a for differentiation into neurons, and with the expression of 04 and CNPOase for differentiation into oligodendrocytes (FIG. 3).

Taken together, the data obtained through this study demonstrate that the Shh plays a critical role in the de-differentiation of mouse astrocytes into neural stem cell-like cells, and that these de-differentiated neural stem cell-like cells can differentiate back into astrocytes, oligodendrocytes, and neurons.

### INDUSTRIAL APPLICABILITY

As described hitherto, Shh is useful in inducing the de-differentiation of astrocytes into neural stem cells, and the de-differentiated neural stem cells can be used in the treatment of various diseases.

### BIBLIOGRAPHY OF PRIOR ART

1. Toru Kondo, Martin Raff. Chromatin remodeling and histone modification in the conversion of oligodendrocyte precursors to neural stem cells. Genes & Development 2004; 18: 2963-2972
2. Alexis Joannides, Phil Gaughwin, Chistof Shwiening, Henry Majed, Jane Sterling, Alastair Compston, Siddharthan Chandran. Efficient generation of neural precursors from adult human skin: astrocytes promote neurogenesis from skin-derived stem cells. Lancet 2004; 363:172-178
3. Sally Temple. The development of neural stem cells. Nature 2001; 414:112-117
4. Hsieh J, Nakashima K, Kuwabara T, Mejia E, Gage F H. Histone deacetylase inhibitor-mediated neuronal differentiation of multipotent adult neural progenitor cells. PNAS 2004; 101:16659-16664
5. Tarasenko Y I, Yu Y, Jordan P M, Bottenstein J, Wu P. Effect of growth factors on proliferation and phenotypic differentiation of human fetal neural stem cells. Journal of Neuroscience Research 2004; 78:625-636
6. Ping Wu, Yevgeniy Tarasenko, Yanping Gu, Li-Yen M. Huang, Richard E. Coggeshall and Yongjia Yu. Region-specific generation of cholinergic neurons from fetal human neural stem cells grafted in adult rat. Nature neuroscience 2005; 5:1271-1278
7. Hu X, Jin L, Feng L. Erk1/2 but not PI3K pathway is required for neurotrophin 3-induced oligodendrocyte differentiation of post-natal neural stem cells. Journal of Neurochemistry 2004; 90:1339-1347
8. Pleasantin Mill, Rong Mo, Ming Chang Hu, Lina Dagnino, Norman D. Rosenblum, Chi-chung Hui. Shh controls epithelial proliferation via independent pathways that converge on N-myc Development 2005; 9:293-33.

1. A composition for inducing de-differentiation of astrocytes into neural stem cells, comprising an Shh (sonic hedgehog) protein or a nucleic acid material containing a nucleotide sequence coding for an Shh protein.

2. The composition as set forth in claim 1, wherein the nucleic acid material containing a nucleotide sequence coding for an Shh protein is a vector that allows the Shh protein to be expressed.

3. The composition as set forth in claim 1, wherein the nucleic acid material containing a nucleotide sequence coding for an Shh protein is a virus that expresses the Shh protein.

4. The composition as set forth in claim 1, wherein the de-differentiated neural stem cells have ability to differentiate into astrocytes, neurons and oligodendrocytes.

5. A method for inducing the de-differentiation of astrocytes into neural stem cells by treating the astrocytes with an Shh protein or a nucleic acid material containing a nucleotide sequence coding for an Shh protein.

6. The method as set forth in claim 5, wherein the de-differentiated neural stem cells have ability to differentiate into astrocytes, neurons and oligodendrocytes.

7. The method as set forth in claim 5, comprising steps of:
(i) culturing astrocytes in a medium;
(ii) treating the astrocytes with the Shh protein or the nucleic acid material; and
(iii) inducing the astrocytes to differentiate into neural stem cells.

8. The medium as set forth in claim 7, wherein the medium of step (i) contains bFGF.

9. A neural stem cell, produced using the method of claim 5.

10. A method of differentiating the neural stem cells produced using the method of claim 5 into astrocytes, neurons, and oligodendrocytes.
